(19) United States
(12) Patent Application Publication
(10) Pub. No.: US 2010/0310541 A1
(43) Pub. Date: Dec. 9, 2010

(54) Compositions and Methods for Reducing the Toxicity of Certain Toxins

(75) Inventors: Marco Kessler, Danvers, MA (US); Mark G. Currie, Sterling, MA (US); Robert Busby, Weymouth, MA (US)

Correspondence Address: Jonathan P. O''Brien, Ph.D.;Honigman Miller Schwartz and Cohn, 350 East Michigan Avenue, Suite 300, KALAMAZOO, MI, 49007, US

(73) Assignee: IRONWOOD PHARMACEUTICALS, INC., CAMBRIDGE, MA (US)

(21) Appl. No.: 12/601,994

(22) PCT Filed: May 27, 2008
(86) PCT No.: PCT/US08/64851

§ 371 Date: Aug. 23, 2010

Related U.S. Application Data
(60) Provisional application No. 60/940,269, filed on May 25, 2007.

(51) Int. Cl. A61K 38/44; A61K 31/095; A61K 38/06; A61K 33/04; A61P 31/04; A61P 31/00; A61P 29/00

(52) U.S. Cl. 424/94.4; 514/706; 514/21.9; 424/702

(57) ABSTRACT

Compositions and methods for reducing the toxic effect of certain peptide toxins by administering an agent that directly or indirectly reduces disulfide bonds that are important for maintaining the toxin in an active conformation. Also described are compositions and methods for reducing the toxic effect of toxins that contain a heavy metal using an agent that destabilizes the binding of a metal ion that is important for toxin activity.

### BACKGROUND

Many toxins, e.g., toxins produced by bacteria, enter the body through the digestive system. Other toxins enter the body through the respiratory system and still others can be absorbed through the skin. In some cases, a microorganism enters the body and produces toxin within the body. Referring to toxins that enter the body through the digestive system, in some cases the toxin itself is ingested and in other cases a microorganism capable of producing the toxin is ingested and the microorganism colonizes the digestive tract where it produces and secretes toxins. For example, botulinum neurotoxin (BoNT) is synthesized and secreted by the anaerobic bacterium Clostridium botulinum and exists in seven related serotypes. BoNT serotype A is a very potent neurotoxin that causes botulism, an often fatal disease characterized by flaccid paralysis. Intoxication with BoNT occurs either by ingestion of a substance, usually a foodstuff, contaminated with BoNT or by ingestion of bacteria that colonize the gut and produce the neurotoxin. In both cases, the toxin escapes the gastrointestinal system to the blood and lymph, eventually reaching the peripheral cholinergic nerve endings that are the target of the toxin's action.

### SUMMARY

Described herein are compositions and methods for reducing the toxic effect of certain toxins. The compositions act by directly or indirectly altering the structure of certain toxins. In some cases, particularly for peptide toxins, the compositions act, directly or indirectly, to reduce disulfide bonds that are important for maintaining the toxin in an active conformation. These disulfide bonds can be interchain disulfide bonds (e.g., in the case of BoNT) or intrachain disulfide bonds in the case of various toxins produced by E. coli. The composition may contain a reducing agent that directly reduces disulfide bonds or it may contain one or more agents that promote a reducing environment, i.e., an environment which increases the rate or extent of reduction of a disulfide bond, within the gastrointestinal tract, for example, by acting together with other agents present in the gastrointestinal tract. In the case of a toxin containing a heavy metal ion that is important for activity, the compositions act, directly or indirectly, to destabilize the binding of a metal ion that is important for toxin activity.

The compositions and methods can be used to treat patients infected with a microorganism that produces a toxin and to treat patients that have ingested or inhaled or have otherwise been exposed to a toxin. The agents can be used to treat patients that have been exposed to a toxin or a toxin-producing microorganism as a result of bioterrorism.

### BRIEF DESCRIPTION OF DRAWINGS

FIG. 1 shows LC/MS data of SEQ ID NO: 1 in its oxidized (a) and reduced and alkylated (b) forms.

FIG. 2 shows mass spectrum data of native (a) and reduced and alkylated (b) SEQ ID NO:1.

FIG. 3 shows the effect of DTT on SEQ ID NO:1 at multiple concentrations.

FIG. 4 is a graphical depiction of the effects of DTT on SEQ ID NO:1.

FIG. 5 shows the effect of DTT on SEQ ID NO:1 at multiple concentrations.

### DETAILED DESCRIPTION

Many peptide toxins are very stable in the human body, including in the gastrointestinal tract. In some cases this stability is conferred, at least in part, by the presences of disulfide bonds (interchain or intrachain or both) which hold the toxin in a compact secondary structure that interferes with proteolysis of the toxin. Reduction of some or all of the disulfide bonds can cause a change in the structure of the peptide that reduces or destroys the activity of the peptide and/or increases proteolysis of the peptide. Some toxins include a heavy metal ion that is important for activity and/or stability. Disruption of the heavy metal binding can reduce the toxicity and/or stability of such a toxin.

It has been found that certain stable peptides containing disulfide bonds are digested in the human intestinal tract by a process that entails reduction of the disulfide bonds and proteolytic digestion (cleavage) of the peptide to peptide fragments and amino acids. Peptide toxins containing disulfide bonds can be rendered more susceptible to proteolytic digestion, for example within the gastrointestinal tract, by exposing the peptide toxin to an agent or agents that will lead, directly or indirectly, to reduction of the disulfide bonds within the peptide toxin. For example, a patient that has been exposed to a toxin or a microorganism that produces a toxin can be treated with a reducing agent capable of reducing a disulfide bond within the toxin. Alternatively, a patient can be treated with an agent or combination of agents that leads to a reducing environment at the locus of the toxin or the locus of toxin production, e.g., within the gastrointestinal tract.

The toxicity of a toxin that is complexed with a heavy metal can be reduced in a somewhat similar manner by exposing them to an agent or agents that directly or indirectly destabilize(s) the interaction between the heavy metal and the remainder of the toxin, thereby destabilizing the toxin structure and reducing or destroying the activity of the toxin.

Agents for Promoting Reduction of Disulfide Bonds Glutathione (2-amino-5-{([2-[(carboxymethyl)amino]-1-(mercaptomethyl)-2-oxoethyl]amino}-5-oxopentanoic acid) is a reducing agent found within many cells. It can exist in a reduced or oxidized form. The reduced form is usually referred to as glutathione, reduced glutathione or GSH. The oxidized form is a dimer that is commonly referred to as oxidized glutathione, glutathione dimer, glutathione disulfide, diglutathione or GSSG.

Exposure of a disulfide bond-containing toxin to GSH should lead to reduction of the disulfide bonds in the toxin. The loss of disulfide bonds is expected to lead to destabilization of the tertiary structure of the toxin and thereby increase the susceptibility of the toxin to proteolytic digestion within the gastrointestinal tract.

In the body, glutaredoxin (Grx) catalyses the reduction of disulfide bonds in proteins thereby converting glutathione (GSH) to glutathione disulfide (GSSG). GSSG is in turn recycled to GSH by glutathione reductase at the expense of NADPH. The human intestinal fluid contains both glutaredoxin and glutathione reductase. By administering Grx, either on its own or together with GSH, it is expected that a more reducing environment would be created in the digestive tract. A more reducing environment is expected to cause greater reduction of disulfide bonds within toxins present in the digestive tract. In some instances it may also be desirable to administer GSSG and/or NADPH together with Grx and/or GSH so that GSH is effectively regenerated.

Additional agents that promote the reduction of disulfide bonds useful in the methods described herein include, but are not limited to: cysteine, mercaptoethanol, 2-mercaptoethanol, 2-mercaptoethylamine, dithioerythritol (DTE), dithiothreitol (DTT), glutathione (e.g. reduced glutathione), Tiopronin, 2-mercaptopropionic acid (2-MPA), n-acetylcysteine (NAC), ascorbic acid, stannous ions/salts (e.g. stannous chloride or stannous tartrate), sodium bisulphite, alkali metal and alkaline earth metal borohydrides (e.g., sodium borohydride), triacetoxyborohydrides, cyanoborohydrides and dithionites (e.g., sodium dithionite), the transition metal salts of transition metals such as zinc, iron, and manganese, and the dimercaptoamides disclosed in US20060013784.

Any reducing agent with acceptably low toxicity can be administered to promote reduction of disulfide bonds in toxins. In some cases the agents for causing reduction of disulfide bonds are formulated so that they are preferentially released in the portion of the digestive system where the toxin is located. Various formulations are discussed in greater detail below.

### Toxins

Exposure to toxins containing disulfide bonds, for example the toxins described below, can be treated by administering an agent described herein that promotes reduction of disulfide bonds. For a review of many peptide toxins see Guidebook to Protein Toxins and Their Use in Cell Biology by Rino Rappuoli and Cesare Montecucco (eds), Oxford University Press 1997.

Bacterial Toxins

Many bacteria and other microorganisms produce toxins that enter the body through the gastrointestinal tract. For example, enterotoxigenic E. coli (including E. coli O157:H7) produce heat-stable enterotoxins and heat-labile enterotoxins that contain disulfide bonds. Among the toxin producing bacteria are: Escherichia coli, Clostridium (e.g., Clostridium dificile, Clostridium perfringes, Clostridium botulinum), Shigella, (e.g., Shigella dysenteriae, Shigella flexneri, Shigella boydii, and Shigella sonnei) Salmonella species (e.g., Salmonella typhimurium).

Tetanus toxin, which is produced by toxigenic strains of Clostridium tetani, is composed of a heavy chain (100 kDa) and a light chain (50 kDa) joined by an interchain disulfide bond. In addition, the carboxy-terminal part of the heavy chain includes an intrachain disulfide bond. Reduction of these disulfide bonds is associated with reduced neurotoxicity (Schaivo et al. 1990 Infect Immun 58: 4136).

Bordetella pertussis colonizes the cilia of the mammalian respiratory epithelium and can cause whooping cough. Bordetella pertussis produces several protein toxins, certain of which contain disulfide bonds.

Pseudomonas aeruginosa, a Gram-negative opportunistic pathogen, is a leading cause of infections in burn victims, immunocompromised individuals, and those suffering from cystic fibrosis. P. aeruginosa produces a number of extracellular toxic products, including, Exotoxin A (ETA). ETA is internalized by a cell surface receptor and is thought to exert cellular toxicity by blocking protein synthesis through ADP ribosylation of translation elongation factor 2 as well as through other mechanisms.

Staphylococcus aureus produces two broad classes of toxins: pyrogenic toxin superantigens (PTSAgs) and hemolysins. PTSAg can cause toxic shock syndrome and staphylococcal food poisoning. Staphylococcal PTSAgs include: toxic shock syndrome toxin-1 (TSST-1) and several staphylococcal enterotoxins (SEs) (SEA, SEB, SEC, SED, SEE, SEG, and SEH). The enterotoxins are quite stable and have an intramolecular disulfide bond.

Staphylococcus aureus is a leading cause of gastroenteritis resulting from the consumption of contaminated food. Staphylococcal food poisoning is due to the absorption of staphylococcal enterotoxin.

Scorpion Toxins

Numerous peptide toxins produced by scorpions are known. Certain of the peptide toxins are specific for sodium channels and others are specific for potassium channels or calcium channels (e.g., toxin from Buthotus hottentota (Valdivia et al. 1991 J Biol Chem 266:19135)) or chloride channels (Lippens et al. 1995 Biochemistry 34:13). At least 120 potassium channel specific scorpion toxins are known. They contain 23 to 64 amino acids and include three or four disulfide bonds. Two of the disulfide bonds covalently link a segment of alpha helix to one strand of a beta-sheet structure. For a review of many scorpion toxins see Rodriguez de la Vega et al. (Toxicon 43:865, 2004; see Table 1); Possani et al. (Eur J. Biochem 264:287, 1999; see Table 1); and Zamudio et al. (1997 FEBS Lett. 405:385).

Spider Toxins

Many peptide toxins present in spider venom, including agatoxins, hanatoxin I, heteropodatoxin I, huwentoxin I (Liang 2004 Toxicon 43:575) and curtoxin I, have multiple disulfide bonds. For a review see Corzo et al. (2003 Cellular and Molecular Life Sciences 60:2409) and Lachlan (2002 Toxicon 40:225).

Cone Snail Toxins

Cone snail toxins are most often fewer than 30 amino acids long, yet contain numerous disulfide bonds. Such toxins include: alpha-conotoxins. alpha-A-conotoxins, delta-conotoxins, mu-conotoxins, omega-conotoxins, O-superfamily conotoxins, mu-O-conotoxins, and toxins from C. textile.

Snake Toxins

Many snake toxins contain one or more disulfide bonds (see Gawade 2004 Journal of Toxicology—Toxin Reviews 23:37 Snake Toxins, Harvey, A. L. (ed) 1991 Pergamon Press, New York; Karlsson (1979) Chemistry of protein toxins in snake venoms. in Lee (ed) Snake venoms, Handbook of Experimental Pharmacology, Springer Verlag, Berlin; and Tamiya et al. 1985 J Biochem (Tokyo) 98:289-303). Among the snake toxins of interest are: curaremimetic toxins, dendrotoxins, kappa toxons, cytotoxins, and fasciculins (such as FAS2, a neurotoxin from green mamba snake venom).

Fungal Toxins

Some non-peptide toxins, such as fungal gliotoxin, include a disulfide bond that may be susceptible to reduction.

### Antibiotics

In many cases intoxication occurs through bacterial infection. Thus, it can be useful to administer an antibiotic together with an agent for promoting reduction of disulfide bonds. Among the antibiotics that can be administered are: Anthracyclines (e.g., Doxorubicin (Dox), Daunorubicin, and Mitoxantrone); Aminoglycosides (e.g., Amikacin, Gentamicin, Kanamycin, Neomycin, Netilmicin, Streptomycin, Tobramycin, Paromornycin, Hygromycin, and Spectinomycin); Carbapenems (e.g., Ertapenem Doripenem, Ertapenem, Faropenem, Imipenem/Cilastatin, Meropenem, and Panipenem/Betamipron); Carbacephems (e.g., Loracarbef); Cephalosporins (e.g., Cefacetrile (Cephacetrile), Cefaclomezine, Cefaclor (Ceclor®, Distaclor®, Keflor®, Raniclor®), Cefadroxil (Cefadroxyl; Duricef®), Cefalexin (Cephalexin; Keflexe), Cefalonium (Cephalonium), Cefaloram, Cefaloridine (Cephaloradinc), Cefalotin (Cephalothin; Keflin®), Cefamandole, Cefaparole, Cefapirin (Cephapirin; Cefadryl®), Cefataxime, Cefatrizine, Cefazaflur, Cefazedone, Cefazolin, Cefazolin (Cephazolin; Ancef®, Kefzol®), Cefcanel, Cefcapene, Cefclidine, Cefdaloxime, Cefdinir (Omnicef®), Cefditoren, Cefedrolor, Cefempidone, Cefepime (Maxipime®), Cefetamet, Cefetrizole, Cefivitril, Cefixime (Suprax®), Cefluprenam, Cefmatilen, Cefinenoxime, Cefinepidium, Cefodizime, Cefonicid (Monocid®), Cefoperazone (Cefobid®), Ceforanide, Cefoselis, Cefotaxime (Claforan®), Cefotiam, Cefovecin, Cefoxazole, Cefozopran, Cefpimizole, Cefpiramide, Cefpirome, Cefpodoxime (Vantin®), Cefprozil (Cefproxil; Cefzil®), Cefquinome, Cefradine (Cephradine; Velosef®), Cefrotil, Cefroxadine, Cefsulodin, Cefsumide, Ceftazidime (Fortum®, Fortaz®), Cefteram, Ceftezole, Ceftibuten (Cedax®), Ceftiofur, Ceftiolene, Ceftioxide, Ceftizoxime (Cefizax®), Ceftobiprole, Ceftobiprole (Previously Bal 5788), Ceftobiprole (Previously Bal 9141 and Ro 63-9141), Ceftriaxone (Rocephin®), Cefuracetime, Cefuroxime (Zinnat®, Zinacef®, Ceftin®, Biofuroksym®), Cefuzonam, and Cephaloglycin); Cephamycins (e.g., Cefbuperazone, Cefinetazole (Zefazone®), Cefminox, Cefotetan (Cefotan®), Cefoxitin (Mefoxin®)); Glycopeptides (e.g., Monobactams (Aztreonam), Teicoplanin, and Vancomycin); Ketolides (e.g., Ansamycin, Carbomycin, Cethromycin, Oleandomycin, Spiramycin, Telithromycin (Ketek®), and Tylocine); Macrolides (e.g., Azithromycin (Zithromax®, Zitromax®), Brefeldin A, Carbomycin A, Chlorothricin, Clarithromycin (Biaxin®), Dirithromycin (Dynabac®), Erythromycin, Fk-506, Josamycin, Kitasamycin, L-865,818, Midecamicine/Midecamicine Acetate, Oleanomycin, Roxithromycin (Rulid®, Surlid®), Spiramycin, Troleandomycin, and Tylosin/Tylocine (Tylan®)); Oxacephems (e.g., Latamoxef (Moxalactam) and Flomoxef); Penicillins (e.g., Amoxicillin, Ampicillin, Azlocillin, Carbenicillin, Cloxacillin, Dicloxacillin, Flucloxacillin, Methicillin, Methicillin, Mezlocillin, Nafcillin, Oxacillin, Piperacillin, Pivampicillin, and Ticarcillin); Polymyxins (e.g., Polymyxin E (Colistin), Polymyxin B, and Surfactin); Quinolones (e.g., Balofloxacin, Cinoxacin (Cinoxacin®), Ciprofloxacin (Cipro®, Ciproxin®), Clinafloxacin, Danofloxacin (Advocin®, Advocid®), Difloxacin (Dicural®, Vetequinon®), Ecinofloxacin, Enoxacin (Enroxil®, Penetrex®), Enrofloxacin (Baytril®), Fleroxacin (Megalone®), Flumequine (Flubactin®), Gatifloxacin (Tequin®, Zymar®), Gemifloxacin (Factive®), Grepafloxacin (Raxar®), Levofloxacin (Cravit®, Levaquin®), Lomefloxacin (Maxaquin®), Marbofloxacin (Marbocyl®, Zenequin®), Moxifloxacin (Avelox®), Nadifloxacin, Nalidixic Acid, Nalidixic Acid (Neggam®, Wintomylon®), Norfloxacin (Noroxin®, Quinabic®, Janacin®), Norfloxin, Ofloxacin (Floxin®, Oxaldin®, Tarivid®), Orbifloxacin (Orbax®, Victas®), Oxolinic Acid, Pazufloxacin Mesilate, Pefloxacin, Pipemidic Acid, Piromidic Acid, Prulifloxacin, Rosoxacin, Rufloxacin, Sarafloxacin (Floxasol®, Saraflox®, Sarafin®), Sitafloxacin, Sparfloxacin (Zagam®), Temafloxacin, Tosufloxacin, and Trovafloxacin (Trovan®)); Rifamycins (e.g., Rifabutin, Rifampicin, Rifapentine, and Rifaximin); Sulfonamides (e.g., Mafenide, Prontosil, Sulfacetamide, Sulfamethizole, Sulfamethoxazole (With Trimethoprim In Co-Trimoxazole), Sulfanilimide, Sulfasalazine, and Sulfisoxazole); Tetracyclines (e.g., Chlortetracycline, Demeclocycline, Doxycycline, Lymecycline, Meclocycline, Methacycline, Minocycline, Oxytetracycline, Rolitetracycline, Tetracycline and Tigecycline); and other antibiotics, including: Arsphenamine (Salvarsan), Bacitracin, Butoconazole, Camptothecin, Capreornycin, Chalcomycin, Chartreusin, Chloramphenicol (Chloromycetin), Chlorotetracyclines, Chrymutasins, Chrysomicin M, Chrysomicin V, Clindamycin (Cleocin), Clomocyclines, Ellipticines, Elsamicin, Ethambutol, Filipins, Fluconazoles, Fosfomycin, Fungichromins, Furazolidone, Fusidic Acid, Gilvocarin, Griseofulvin, Griseoviridin, Guamecyclines, Ilosamides (I.E. Lincomycin, Clindamycin), Isoniazid, Itraconazoles, Lankacidin-Group Antibiotics (I.E. Lankamycin), Linezolid (Zyvox), Metronidazole (Flagyl), Mitomycin, Mupirocin, Nitrofurantoin (Macrodantin, Macrobid), Nystatins, Phosphomycin, Platensimycin, Pyrazinamide, Quinupristin/Dalfopristin (Syncercid), Ravidomycin, Rifampin, Ristocetins A and B, Spectinomycin, Telithromycin, Teramycins, Tyrothricin, and Wortmannins.

Administration of Agents that Promote Reduction of Disulfide Bonds

The agents described herein for reducing toxicity by promoting reduction of disulfide bonds can be administered in any convenient manner. Where the toxin is found within the gastrointestinal tract, the agent is preferably administered orally, e.g., as composition containing a predetermined amount of the active ingredient as a tablet, cachet, pellet, gel, paste, syrup, bolus, electuary, slurry, sachet, capsule, powder, lyophilized powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. For example, the agent can be formulated so that is it preferentially released in the large intestine. The agents for reducing toxicity can be co-administered with other agents, for example, an antibiotic or other agent intended to combat infection by an infectious agent that produces the toxin or an agent which is intended to treat one or more symptoms caused by the toxin.

The agents described herein can be administered alone or in combination with other agents. For example, the agents for reducing toxicity can be administered together with an agent for treating infection, e.g., an antibiotic or an analgesic compound.

Combination therapy can be achieved by administering two or more agents, e.g., an agent for reducing toxicity described herein and antibiotic, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

Combination therapy can also include the administration of two or more agents via different routes or locations. For example, (a) one agent is administered orally (e.g., a reducing agent) and another agents is administered intravenously (an antibiotic) or (b) one agent is administered orally and another is administered locally. In each case, the agents can either simultaneously or sequentially. Approximate dosages for antibiotics, analgesics and other agents that can be used in combination with agents for promoting reduction of disulfide bonds can be found in standard formularies and other drug prescribing directories. For some drugs, the customary prescribed dose for an indication will vary somewhat from country to country.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose (e.g. celphere, Celphere Beads®), diluents, lubricants, binders, disintegrating agents, and the like), etc. If desired, tablet dosages of the disclosed compositions may be coated by standard aqueous or nonaqueous techniques.

Compositions containing agents for promoting reduction of disulfide bonds may also optionally include other therapeutic ingredients, anti-caking agents, preservatives, sweetening agents, colorants, flavors, desiccants, plasticizers, dyes, glidants, anti-adherents, anti-static agents, surfactants (wetting agents), anti-oxidants, film-coating agents, and the like. Any such optional ingredient must be compatible with the compound of the invention to insure the stability of the formulation. The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: corn starch, potato starch, other starches, gelatin, natural and synthetic gums such as acacia, xanthan, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., povidone, crospovidone, copovidone, etc), methyl cellulose, Methocel, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof,

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, dextrose, fructose, honey, lactose anhydrate, lactose monohydrate, lactose and aspartame, lactose and cellulose, lactose and microcrystalline cellulose, maltodextrin, maltose, mannitol, microcrystalline cellulose & guar gum, molasses, sucrose, or mixtures thereof,

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums (like gellan), low-substituted hydroxypropyl cellulose, or mixtures thereof,

LUBRICANTS: calcium stearate, magnesium stearate, mineral oil, light mineral oil, glycerin, sorbitol, mannitol, polyethylene glycol, other glycols, stearic acid, sodium lauryl sulfate, sodium stearyl fumarate, vegetable based fatty acids lubricant, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof,

ANTI-CAKING AGENTS: calcium silicate, magnesium silicate, silicon dioxide, colloidal silicon dioxide, talc, or mixtures thereof,

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and

COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

The formulation can also include other excipients and categories thereof including but not limited to L-histidine, Pluronic®, Poloxamers (such as Lutrol® and Poloxamer 188), ascorbic acid, glutathione, permeability enhancers (e.g. lipids, sodium cholate, acylcarnitine, salicylates, mixed bile salts, fatty acid micelles, chelators, fatty acid, surfactants, medium chain glycerides), protease inhibitors (e.g. soybean trypsin inhibitor, organic acids), pH lowering agents and absorption enhancers effective to promote bioavailability (including but not limited to those described in U.S. Pat. No. 6,086,918 and U.S. Pat. No. 5,912,014), creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD& C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No. 10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

Solid oral dosage forms may optionally be treated with coating systems (e.g. Opadry® a film coating system, for example Opadry® blue (OY-LS-20921), Opadry® white (YS-2-7063), Opadry® white (YS-1-7040), and black ink (S-1-8106).

The agents either in their free form or as a salt can be combined with a polymer such as polylactic-glycolic acid (PLGA), poly-(1)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. 20030068384) to create a sustained release formulation. Such formulations can be used to implants that release a peptide or another agent over a period of a few days, a few weeks or several months depending on the polymer, the particle size of the polymer, and the size of the implant (see, e.g., U.S. Pat. No. 6,620,422). Other sustained release formulations and polymers for use in are described in EP 0 467 389 A2, WO 93/24150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and. US20020019446. In such sustained release formulations microparticles (Delie and Blanco-Prieto 2005 Molecule 10:65-80) of peptide are combined with microparticles of polymer. One or more sustained release implants can be placed in the large intestine, the small intestine or both. U.S. Pat. No. 6,011,011 and WO 94/06452 describe a sustained release formulation providing either polyethylene glycols (i.e. PEG 300 and PEG 400) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. Pat. No. 5,877,224.

In some cases the agents for reducing toxicity can be administered, e.g., by intravenous injection, intramuscular injection, subcutaneous injection, intraperitoneal injection, topical, sublingual, intraarticular (in the joints), intradermal, buccal, ophthalmic (including intraocular), intranasally (including using a cannula), intraspinally, intrathecally, or by other routes. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115-124)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasally using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

Where the toxin is found in the airways, e.g., the lungs, the agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation.

Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluorocarbon propellants such as a mixture of Propellants 11, 12, and/or 114), propane, nitrogen, and the like. Pulmonary formulations may include permeation enhancers such as fatty acids, saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C₈-C₁₆ fatty acid salt, a bile salt, a phospholipid, or alkyl saccharide are advantageous in that some of them also reportedly enhance absorption of the peptide in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U.S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The agents for reducing toxicity can be incorporated into pharmaceutically-acceptable nanoparticle, nanosphere, and nanocapsule formulations (Delie and Blanco-Prieto 2005 Molecule 10:65-80). Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, ultrafine particles (sized around 0.1 μm) can be designed using polymers able to be degraded in vivo (e.g. biodegradable polyalkyl-cyanoacrylate nanoparticles). Such particles are described in the prior art (Couvreur et al, 1980; 1988; zur Muhlen et al., 1998; Zambaux et al. 1998; Pinto-Alphandry et al., 1995 and U.S. Pat. No. 5,145,684).

The agents for reducing toxicity can be formulated with pH sensitive materials which may include those described in WO04041195 (including the seal and enteric coating described therein) and pH-sensitive coatings that achieve delivery in the colon including those described in U.S. Pat. No. 4,910,021 and WO9001329. U.S. Pat. No. 4,910,021 describes using a pH-sensitive material to coat a capsule. WO9001329 describes using pH-sensitive coatings on beads containing acid, where the acid in the bead core prolongs dissolution of the pH-sensitive coating. U.S. Pat. No. 5,175,003 discloses a dual mechanism polymer mixture composed of pH-sensitive enteric materials and film-forming plasticizers capable of conferring permeability to the enteric material, for use in drug-delivery systems; a matrix pellet composed of a dual mechanism polymer mixture permeated with a drug and sometimes covering a pharmaceutically neutral nucleus; a membrane-coated pellet comprising a matrix pellet coated with a dual mechanism polymer mixture envelope of the same or different composition; and a pharmaceutical dosage form containing matrix pellets. The matrix pellet releases acid-soluble drugs by diffusion in acid pH and by disintegration at pH levels of nominally about 5.0 or higher. The agents of the invention may be formulated in the pH triggered targeted control release systems described in WO04052339. The agents of the invention may be formulated according to the methodology described in any of WO03105812 (extruded hydratable polymers); WO0243767 (enzyme cleavable membrane translocators); WO03007913 and WO03086297 (mucoadhesive systems); WO02072075 (bilayer laminated formulation comprising pH lowering agent and absorption enhancer); WO04064769 (amidated peptides); WO05063156 (solid lipid suspension with pseudotropic and/or thixotropic properties upon melting); WO03035029 and WO03035041 (erodible, gastric retentive dosage forms); U.S. Pat. No. 5,007,790 and U.S. Pat. No. 5,972,389 (sustained release dosage forms); WO04112711 (oral extended release compositions); WO05027878, WO02072033, and WO02072034 (delayed release compositions with natural or synthetic gum); WO05030182 (controlled release formulations with an ascending rate of release); WO05048998 (microencapsulation system); U.S. Pat. No. 5,952,314 (biopolymer); U.S. Pat. No. 5,108,758 (glassy amylose matrix delivery); U.S. Pat. No. 5,840,860 (modified starch based delivery). JP10324642 (delivery system comprising chitosan and gastric resistant material such as wheat gliadin or zein); U.S. Pat. No. 5,866,619 and U.S. Pat. No. 6,368,629 (saccharide containing polymer); U.S. Pat. No. 6,531,152 (describes a drug delivery system containing a water soluble core (Ca pectinate or other water-insoluble polymers) and outer coat which bursts (eg hydrophobic polymer-Eudragrit)); U.S. Pat. No. 6,234,464; U.S. Pat. No. 6,403,130 (coating with polymer containing casein and high methoxy pectin; WO0174175 (Maillard reaction product); WO05063206 (solubility increasing formulation); WO04019872 (transferring fusion proteins). The agents of the invention may be formulated using gastrointestinal retention system technology (GIRES; Merrion Pharmaceuticals). GIRES comprises a controlled-release dosage form inside an inflatable pouch, which is placed in a drug capsule for oral administration. Upon dissolution of the capsule, a gas-generating system inflates the pouch in the stomach where it is retained for 16-24 hours, all the time releasing agents of the invention.

The agents for reducing toxicity can be formulated in an osmotic device including the ones disclosed in U.S. Pat. No. 4,503,030, U.S. Pat. No. 5,609,590 and U.S. Pat. No. 5,358,502. U.S. Pat. No. 4,503,030 discloses an osmotic device for dispensing a drug to certain pH regions of the gastrointestinal tract. More particularly, the invention relates to an osmotic device comprising a wall formed of a semi-permeable pH sensitive composition that surrounds a compartment containing a drug, with a passageway through the wall connecting the exterior of the device with the compartment. The device delivers the drug at a controlled rate in the region of the gastrointestinal tract having a pH of less than 3.5, and the device self-destructs and releases all its drug in the region of the gastrointestinal tract having a pH greater than 3.5, thereby providing total availability for drug absorption. U.S. Pat. No. 5,609,590 and U.S. Pat. No. 5,358,502 disclose an osmotic bursting device for dispensing a beneficial agent to an aqueous environment. The device comprises a beneficial agent and osmagent surrounded at least in part by a semi-permeable membrane. The beneficial agent may also function as the osmagent. The semi-permeable membrane is permeable to water and substantially impermeable to the beneficial agent and osmagent. A trigger means is attached to the semi-permeable membrane (e.g., joins two capsule halves). The trigger means is activated by a pH of from 3 to 9 and triggers the eventual, but sudden, delivery of the beneficial agent. These devices enable the pH-triggered release of the beneficial agent core as a bolus by osmotic bursting.

The agents for reducing toxicity may be formulated as described in U.S. Pat. No. 5,316,774 which discloses a composition for the controlled release of an active substance comprising a polymeric particle matrix, where each particle defines a network of internal pores. The active substance is entrapped within the pore network together with a blocking agent having physical and chemical characteristics selected to modify the release rate of the active substance from the internal pore network. In one embodiment, drugs may be selectively delivered to the intestines using an enteric material as the blocking agent. The enteric material remains intact in the stomach but degrades under the pH conditions of the intestines. In another embodiment, the sustained release formulation employs a blocking agent, which remains stable under the expected conditions of the environment to which the active substance is to be released. The use of pH-sensitive materials alone to achieve site-specific delivery is difficult because of leaking of the beneficial agent prior to the release site or desired delivery time and it is difficult to achieve long time lags before release of the active ingredient after exposure to high pH (because of rapid dissolution or degradation of the pH-sensitive materials).

The agents for reducing toxicity may also be formulated in a hybrid system which combines pH-sensitive materials and osmotic delivery systems. These hybrid devices provide delayed initiation of sustained-release of the beneficial agent. In one device a pH-sensitive matrix or coating dissolves releasing osmotic devices that provide sustained release of the beneficial agent see U.S. Pat. Nos. 4,578,075, 4,681,583, and 4,851,231. A second device consists of a semipermeable coating made of a polymer blend of an insoluble and a pH-sensitive material. As the pH increases, the permeability of the coating increases, increasing the rate of release of beneficial agent see U.S. Pat. Nos. 4,096,238, 4,503,030, 4,522,625, and 4,587,117.

The agents for reducing toxicity may be formulated in terpolumers according to U.S. Pat. No. 5,484,610 which discloses terpolymers which are sensitive to pH and temperature which are useful carriers for conducting bioactive agents through the gastric juices of the stomach in a protected form. The terpolymers swell at the higher physiologic pH of the intestinal tract causing release of the bioactive agents into the intestine. The terpolymers are linear and are made up of 35 to 99 wt % of a temperature sensitive component, which imparts to the terpolymer LCST (lower critical solution temperature) properties below body temperatures, 1 to 30 wt % of a pH sensitive component having a pKa in the range of from 2 to 8 which functions through ionization or deionization of carboxylic acid groups to prevent the bioactive agent from being lost at low pH but allows bioactive agent release at physiological pH of about 7.4 and a hydrophobic component which stabilizes the LCST below body temperatures and compensates for bioactive agent effects on the terpolymers. The terpolymers provide for safe bioactive agent loading, a simple procedure for dosage form fabrication and the terpolymer functions as a protective carrier in the acidic environment of the stomach and also protects the bioactive agents from digestive enzymes until the bioactive agent is released in the intestinal tract.

The agents for reducing toxicity may be formulated in pH sensitive polymers according to those described in U.S. Pat. No. 6,103,865. U.S. Pat. No. 6,103,865 discloses pH-sensitive polymers containing sulfonamide groups, which can be changed in physical properties, such as swellability and solubility, depending on pH and which can be applied for a drug-delivery system, bio-material, sensor, and the like, and a preparation method therefore. The pH-sensitive polymers are prepared by introduction of sulfonamide groups, various in pKa, to hydrophilic groups of polymers either through coupling to the hydrophilic groups of polymers, such as acrylamide, N,N-dimethylacrylamide, acrylic acid, N-isopropylacrylamide and the like or copolymerization with other polymerizable monomers. These pH-sensitive polymers may have a structure of linear polymer, grafted copolymer, hydrogel or interpenetrating network polymer.

The agents for reducing toxicity may be formulated according U.S. Pat. No. 5,656,292 which discloses a composition for pH dependent or pH regulated controlled release of active ingredients especially drugs. The composition consists of a compactable mixture of the active ingredient and starch molecules substituted with acetate and dicarboxylate residues. The preferred dicarboxylate acid is succinate. The average substitution degree of the acetate residue is at least 1 and 0. 2-1. 2 for the dicarboxylate residue. The starch molecules can have the acetate and dicarboxylate residues attached to the same starch molecule backbone or attached to separate starch molecule backbones. The present invention also discloses methods for preparing said starch acetate dicarboxylates by transesterification or mixing of starch acetates and starch dicarboxylates respectively.

The agents for reducing toxicity may be formulated according to the methods described in U.S. Pat. Nos. 5,554,147, 5,788,687, and 6,306,422 which disclose a method for the controlled release of a biologically active agent wherein the agent is released from a hydrophobic, pH-sensitive polymer matrix. The polymer matrix swells when the environment reaches pH 8.5, releasing the active agent. A polymer of hydrophobic and weakly acidic comonomers is disclosed for use in the controlled release system. Also disclosed is a specific embodiment in which the controlled release system may be used. The pH-sensitive polymer is coated onto a latex catheter used in ureteral catheterization. A ureteral catheter coated with a pH-sensitive polymer having an antibiotic or urease inhibitor trapped within its matrix will release the active agent when exposed to high pH urine.

The agents for reducing toxicity may be formulated in/with bioadhesive polymers according to U.S. Pat. No. 6,365,187. Bioadhesive polymers in the form of, or as a coating on, microcapsules containing drugs or bioactive substances which may serve for therapeutic, or diagnostic purposes in diseases of the gastrointestinal tract, are described in U.S. Pat. No. 6,365,187. The polymeric microspheres all have a bioadhesive force of at least 11 mN/cm² (110 N/m2) Techniques for the fabrication of bioadhesive microspheres, as well as a method for measuring bioadhesive forces between microspheres and selected segments of the gastrointestinal tract in vitro are also described. This quantitative method provides a means to establish a correlation between the chemical nature, the surface morphology and the dimensions of drug-loaded microspheres on one hand and bioadhesive forces on the other, allowing the screening of the most promising materials from a relatively large group of natural and synthetic polymers which, from theoretical consideration, should be used for making bioadhesive microspheres. Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means. The agent can be fused to immunoglobulins or albumin, albumin variants or fragments thereof, or incorporated into a liposome to improve half-life. Thus the peptides described herein may be fused directly or via a peptide linker, water soluble polymer, or prodrug linker to albumin or an analog, fragment, or derivative thereof. Generally, the albumin proteins that are part of the fusion proteins of the present invention may be derived from albumin cloned from any species, including human. Human serum albumin (HSA) consists of a single non-glycosylated polypeptide chain of 585 amino acids with a formula molecular weight of 66,500. The amino acid sequence of human HSA is known [See Meloun, et al. (1975) FEBS Letters 58:136; Behrens, et al. (1975) Fed. Proc. 34:591; Lawn, et al. (1981) Nucleic Acids Research 9:6102-6114; Minghetti, et al. (1986) J. Biol. Chem. 261:6747, each of which are incorporated by reference herein]. A variety of polymorphic variants as well as analogs and fragments of albumin have been described. [See Weitkamp, et al., (1973) Ann. Hum. Genet. 37:219]. For example, in EP 322,094, various shorter forms of HSA. Some of these fragments of HSA are disclosed, including HSA(1-373), HSA(1-388), HSA(1-389), HSA(1-369), and HSA(1-419) and fragments between 1-369 and 1-419. EP 399,666 discloses albumin fragments that include HSA(1-177) and HSA(1-200) and fragments between HSA(1-177) and HSA(1-200). Methods related to albumin fusion proteins can be found in U.S. Pat. No. 7,056,701, U.S. Pat. No. 6,994,857, U.S. Pat. No. 6,946,134, U.S. Pat. No. 6,926,898, and U.S. Pat. No. 6,905,688 and the related priority documents and references cited therein. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. Peptides can also be modified with alkyl groups (e.g., C₁-C₂₀ straight or branched alkyl groups); fatty acid radicals; and combinations of PEG, alkyl groups and fatty acid radicals (see U.S. Pat. No. 6,309,633; Soltero et al., 2001 Innovations in Pharmaceutical Technology 106-110). The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

The agents for reducing toxicity can be administered using COLAL® colonic drug delivery technology (U.S. Pat. No. 6,534,549) BTGInternational, Ltd.; Alizyme, plc; Cambridge, UK) in which small pellets containing the agents are coated with ethylcellulose and a specific form of amylose. This coating prevents drug release in the stomach and small intestine. When the pellets reach the colon the amylose in the coating is broken down by bacterial enzymes and the agent is released.

Matrix devices are a common device for controlling the release of various agents. In such devices, the agents described herein are generally present as a dispersion within the polymer matrix, and are typically formed by the compression of a polymer/drug mixture or by dissolution or melting. The dosage release properties of these devices may be dependent upon the solubility of the agent in the polymer matrix or, in the case of porous matrices, the solubility in the sink solution within the pore network, and the tortuosity of the network. In one instance, when utilizing an erodible polymeric matrix, the matrix imbibes water and forms an aqueous-swollen gel that entraps the agent. The matrix then gradually erodes, swells, disintegrates or dissolves in the GI tract, thereby controlling release of one or more of the agents described herein. In non-erodible devices, the agent is released by diffusion through an inert matrix.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use.

The erodible polymeric matrix into which an agent described herein can be incorporated may generally be described as a set of excipients that are mixed with the agent following its formation that, when contacted with the aqueous environment of use imbibes water and forms a water-swollen gel or matrix that entraps the drug form. Drug release may occur by a variety of mechanisms, for example, the matrix may disintegrate or dissolve from around particles or granules of the agent or the agent may dissolve in the imbibed aqueous solution and diffuse from the tablet, beads or granules of the device. One ingredient of this water-swollen matrix is the water-swellable, erodible, or soluble polymer, which may generally be described as an osmopolymer, hydrogel or water-swellable polymer. Such polymers may be linear, branched, or crosslinked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 daltons, for example, the Dow Methocel™ series E5, E15LV, E50LV and K100LY) and high viscosity (MW greater than 50,000 daltons, for example, E4MCR, E10MCR, K4M, K15M and K100M and the Methocel™ K series) HPMC. Other commercially available types of HPMC include the Shin Etsu Metolose 90SH series. The choice of matrix material can have a large effect on the maximum drug concentration attained by the device as well as the maintenance of a high drug concentration. The matrix material can be a concentration-enhancing polymer, for example, as described in WO05/011634.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

The erodible matrix polymer may contain a wide variety of the same types of additives and excipients known in the pharmaceutical arts, including osmopolymers, osmagens, solubility-enhancing or -retarding agents and excipients that promote stability or processing of the device.

Alternatively, the agents for reducing toxicity may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g methyl acrylate-methyl methacrylate copolymers, polyvinyl chloride, polyethylene), hydrophilic polymers (e.g. ethyl cellulose, cellulose acetate, crosslinked polyvinylpyrrolidone (also known as crospovidone)), and fatty compounds (e.g. carnauba wax, microcrystalline wax, and triglycerides). Such devices are described further in Remington: The Science and Practice of Pharmacy, 20th edition (2000).

Matrix controlled release devices may be prepared by blending an agent described herein and other excipients together, and then forming the blend into a tablet, caplet, pill, or other device formed by compressive forces. Such compressed devices may be formed using any of a wide variety of presses used in the fabrication of pharmaceutical devices. Examples include single-punch presses, rotary tablet presses, and multilayer rotary tablet presses, all well known in the art. See for example, Remington: The Science and Practice of Pharmacy, 20th Edition, 2000. The compressed device may be of any shape, including round, oval, oblong, cylindrical, or triangular. The upper and lower surfaces of the compressed device may be flat, round, concave, or convex.

In certain embodiments, when formed by compression, the device has a strength of at least 5 Kiloponds (Kp)/cm² (for example, at least 7 Kp/cm²). Strength is the fracture force, also known as the tablet hardness required to fracture a tablet formed from the materials, divided by the maximum cross-sectional area of the tablet normal to that force. The fracture force may be measured using a Schleuniger Tablet Hardness Tester, Model 6D. The compression force required to achieve this strength will depend on the size of the tablet, but generally will be greater than about 5 kP/cm². Friability is a well-know measure of a device's resistance to surface abrasion that measures weight loss in percentage after subjecting the device to a standardized agitation procedure. Friability values of from 0.8 to 1.0% are regarded as constituting the upper limit of acceptability. Devices having a strength of greater than 5 kP/cm² generally are very robust, having a friability of less than 0.5%. Other methods for forming matrix controlled-release devices are well known in the pharmaceutical arts. See for example, Remington: The Science and Practice of Pharmacy, 20th Edition, 2000.

As noted above, the agents described herein may also be incorporated into an osmotic control device. Such devices generally include a core containing one or more agents as described herein and a water permeable, non-dissolving and non-eroding coating surrounding the core which controls the influx of water into the core from an aqueous environment of use so as to cause drug release by extrusion of some or all of the core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head).

Osmotic agents create a driving force for transport of water from the environment of use into the core of the device. Osmotic agents include but are not limited to water-swellable hydrophilic polymers, and osmogens (or osmagens). Thus, the core may include water-swellable hydrophilic polymers, both ionic and nonionic, often referred to as osmopolymers and hydrogels. The amount of water-swellable hydrophilic polymers present in the core may range from about 5 to about 80 wt % (including for example, 10 to 50 wt %). Nonlimiting examples of core materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly (methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolat. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The core may include a wide variety of additives and excipients that enhance the performance of the dosage form or that promote stability, tableting or processing. Such additives and excipients include tableting aids, surfactants, water-soluble polymers, pH modifiers, fillers, binders, pigments, disintegrants, antioxidants, lubricants and flavorants. Nonlimiting examples of additives and excipients include but are not limited to those described elsewhere herein as well as microcrystalline cellulose, metallic salts of acids (e.g. aluminum stearate, calcium stearate, magnesium stearate, sodium stearate, zinc stearate), pH control agents (e.g. buffers, organic acids, organic acid salts, organic and inorganic bases), fatty acids, hydrocarbons and fatty alcohols (e.g. stearic acid, palmitic acid, liquid paraffin, stearyl alcohol, and palmitol), fatty acid esters (e.g. glyceryl (mono- and di-) stearates, triglycerides, glyceryl (palmiticstearic) ester, sorbitan esters (e.g. sorbitan monostearate, saccharose monostearate, saccharose monopalmitate, sodium stearyl fumarate), polyoxyethylene sorbitan esters), surfactants (e.g. alkyl sulfates (e.g. sodium lauryl sulfate, magnesium lauryl sulfate), polymers (e.g. polyethylene glycols, polyoxyethylene glycols, polyoxyethylene, polyoxypropylene ethers, including copolymers thereof), polytetrafluoroethylene), and inorganic materials (e.g. talc, calcium phosphate), cyclodextrins, sugars (e.g. lactose, xylitol), sodium starch glycolate). Nonlimiting examples of disintegrants are sodium starch glycolate (e. g., Explotab™ CLV, (microcrystalline cellulose (e.g., Avicel™), microcrystalline silicified cellulose (e.g., ProSolv™), croscarmellose sodium (e.g., Ac-Di-Sol™). When the agent described herein is a solid amorphous dispersion formed by a solvent process, such additives may be added directly to the spray-drying solution when forming an agent described herein/concentration-enhancing polymer dispersion such that the additive is dissolved or suspended in the solution as a slurry, Alternatively, such additives may be added following the spray-drying process to aid in forming the final controlled release device.

A non-limiting example of an osmotic device consists of one or more drug layers containing an agent described herein, such as a solid amorphous drug/polymer dispersion, and a sweller layer that comprises a water-swellable polymer, with a coating surrounding the drug layer and sweller layer. Each layer may contain other excipients such as tableting aids, osmagents, surfactants, water-soluble polymers and water-swellable polymers.

Such osmotic delivery devices may be fabricated in various geometries including bilayer (wherein the core comprises a drug layer and a sweller layer adjacent to each other), trilayer (wherein the core comprises a sweller layer sandwiched between two drug layers) and concentric (wherein the core comprises a central sweller agent surrounded by the drug layer). The coating of such a tablet comprises a membrane permeable to water but substantially impermeable to drug and excipients contained within. The coating contains one or more exit passageways or ports in communication with the drug-containing layer(s) for delivering the drug agent. The drug-containing layer(s) of the core contains the drug agent (including optional osmagents and hydrophilic water-soluble polymers), while the sweller layer consists of an expandable hydrogel, with or without additional osmotic agents.

When placed in an aqueous medium, the tablet imbibes water through the membrane, causing the agent to form a dispensable aqueous agent, and causing the hydrogel layer to expand and push against the drug-containing agent, forcing the agent out of the exit passageway. The agent can swell, aiding in forcing the drug out of the passageway. Drug can be delivered from this type of delivery system either dissolved or dispersed in the agent that is expelled from the exit passageway.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

Other materials useful in forming the drug-containing agent, in addition to the agent described herein itself, include HPMC, PEO and PVP and other pharmaceutically acceptable carriers. In addition, osmagents such as sugars or salts, including but not limited to sucrose, lactose, xylitol, mannitol, or sodium chloride, may be added. Materials which are useful for forming the hydrogel layer include sodium CMC, PEO (e.g. polymers having an average molecular weight from about 5,000,000 to about 7,500,000 daltons), poly(acrylic acid), sodium (polyacrylate), sodium croscarmellose, sodium starch glycolat, PVP, crosslinked PVP, and other high molecular weight hydrophilic materials.

In the case of a bilayer geometry, the delivery port(s) or exit passageway(s) may be located on the side of the tablet containing the drug agent or may be on both sides of the tablet or even on the edge of the tablet so as to connect both the drug layer and the sweller layer with the exterior of the device. The exit passageway(s) may be produced by mechanical means or by laser drilling, or by creating a difficult-to-coat region on the tablet by use of special tooling during tablet compression or by other means.

The osmotic device can also be made with a homogeneous core surrounded by a semipermeable membrane coating, as in U.S. Pat. No. 3,845,770. The agent described herein can be incorporated into a tablet core and a semipermeable membrane coating can be applied via conventional tablet-coating techniques such as using a pan coater. A drug delivery passageway can then be formed in this coating by drilling a hole in the coating, either by use of a laser or mechanical means. Alternatively, the passageway may be formed by rupturing a portion of the coating or by creating a region on the tablet that is difficult to coat, as described above. In one embodiment, an osmotic device comprises: (a) a single-layer compressed core comprising: (i) an agent described herein, (ii) a hydroxyethylcellulose, and (iii) an osmagent, wherein the hydroxyethylcellulose is present in the core from about 2.0% to about 35% by weight and the osmagent is present from about 15% to about 70% by weight; (b) a water-permeable layer surrounding the core; and (c) at least one passageway within the water-permeable layer (b) for delivering the drug to a fluid environment surrounding the tablet. In certain embodiments, the device is shaped such that the surface area to volume ratio (of a water-swollen tablet) is greater than 0.6 mm⁻¹ (including, for example, greater than 1.0 mm⁻¹). The passageway connecting the core with the fluid environment can be situated along the tablet band area. In certain embodiments, the shape is an oblong shape where the ratio of the tablet tooling axes, i.e., the major and minor axes which define the shape of the tablet, are between 1.3 and 3 (including, for example, between 1.5 and 2.5). In one embodiment, the combination of the agent described herein and the osmagent have an average ductility from about 100 to about 200 Mpa, an average tensile strength from about 0.8 to about 2.0 Mpa, and an average brittle fracture index less than about 0.2. The single-layer core may optionally include a disintegrant, a bioavailability enhancing additive, and/or a pharmaceutically acceptable excipient, carrier or diluent.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Nonlimiting examples of standard disintegrants include materials such as sodium starch glycolate (e.g., Explotab™ CLV), microcrystalline cellulose (e.g., Avicel™), microcrystalline silicified cellulose (e.g., ProSoIv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 50-74% of the core agent.

Water-soluble polymers are added to keep particles of the agent suspended inside the device before they can be delivered through the passageway(s) (e.g., an orifice). High viscosity polymers are useful in preventing settling. However, the polymer in combination with the agent is extruded through the passageway(s) under relatively low pressures. At a given extrusion pressure, the extrusion rate typically slows with increased viscosity. Certain polymers in combination with particles of the agent described herein form high viscosity solutions with water but are still capable of being extruded from the tablets with a relatively low force. In contrast, polymers having a low weight-average, molecular weight (<about 300,000) do not form sufficiently viscous solutions inside the tablet core to allow complete delivery due to particle settling. Settling of the particles is a problem when such devices are prepared with no polymer added, which leads to poor drug delivery unless the tablet is constantly agitated to keep the particles from settling inside the core. Settling is also problematic when the particles are large and/or of high density such that the rate of settling increases.

In certain embodiments, the water-soluble polymers for such osmotic devices do not interact with the drug. In certain embodiments the water-soluble polymer is a non-ionic polymer. A nonlimiting example of a non-ionic polymer forming solutions having a high viscosity yet still extrudable at low pressures is Natrosol™ 250H (high molecular weight hydroxyethylcellulose, available from Hercules Incorporated, Aqualon Division, Wilmington, Del.; MW equal to about 1 million daltons and a degree of polymerization equal to about 3,700). Natrosol 250H™ provides effective drug delivery at concentrations as low as about 3% by weight of the core when combined with an osmagent. Natrosol 250H™ NF is a high-viscosity grade nonionic cellulose ether that is soluble in hot or cold water. The viscosity of a 1% solution of Natrosol 250H using a Brookfield LVT (30 rpm) at 25° C. is between about 1, 500 and about 2,500 cps.

In certain embodiments, hydroxyethylcellulose polymers for use in these monolayer osmotic tablets have a weight-average, molecular weight from about 300,000 to about 1.5 million. The hydroxyethylcellulose polymer is typically present in the core in an amount from about 2.0% to about 35% by weight.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

The osmotic controlled release devices can also comprise a coating. In certain embodiments, the osmotic controlled release device coating exhibits one or more of the following features: is water-permeable, has at least one port for the delivery of drug, and is non-dissolving and non-eroding during release of the drug formulation, such that drug is substantially entirely delivered through the delivery port(s) or pores as opposed to delivery primarily via permeation through the coating material itself. Delivery ports include any passageway, opening or pore whether made mechanically, by laser drilling, by pore formation either during the coating process or in situ during use or by rupture during use. In certain embodiments, the coating is present in an amount ranging from about 5 to 30 wt % (including, for example, 10 to 20 wt %) relative to the core weight.

One form of coating is a semipermeable polymeric membrane that has the port(s) formed therein either prior to or during use. Thickness of such a polymeric membrane may vary between about 20 and 800 μm (including, for example, between about 100 to 500 μm). The diameter of the delivery port (s) may generally range in size from 0.1 to 3000 μm or greater (including, for example, from about 50 to 3000 μm in diameter). Such port(s) may be formed post-coating by mechanical or laser drilling or may be formed in situ by rupture of the coatings; such rupture may be controlled by intentionally incorporating a relatively small weak portion into the coating. Delivery ports may also be formed in situ by erosion of a plug of water-soluble material or by rupture of a thinner portion of the coating over an indentation in the core. In addition, delivery ports may be formed during coating, as in the case of asymmetric membrane coatings of the type disclosed in U.S. Pat. No. 5,612,059 and U.S. Pat. No. 5,698,220. The delivery port may be formed in situ by rupture of the coating, for example, when a collection of beads that may be of essentially identical or of a variable agent are used. Drug is primarily released from such beads following rupture of the coating and, following rupture, such release may be gradual or relatively sudden. When the collection of beads has a variable agent, the agent may be chosen such that the beads rupture at various times following administration, resulting in the overall release of drug being sustained for a desired duration.

Coatings may be dense, microporous or asymmetric, having a denser region supported by a thick porous region such as those disclosed in U.S. Pat. No. 5,612,059 and U.S. Pat. No. 5,698,220. When the coating is dense the coating can be composed of a water-permeable material. When the coating is porous, it may be composed of either a water-permeable or a water-impermeable material. When the coating is composed of a porous water-impermeable material, water permeates through the pores of the coating as either a liquid or a vapor. Nonlimiting examples of osmotic devices that utilize dense coatings include U.S. Pat. No. 3,995,631 and U.S. Pat. No. 3,845,770. Such dense coatings are permeable to the external fluid such as water and may be composed of any of the materials mentioned in these patents as well as other water-permeable polymers known in the art.

The membranes may also be porous as disclosed, for example, in U.S. Pat. No. 5,654,005 and U.S. Pat. No. 5,458,887 or even be formed from water-resistant polymers. U.S. Pat. No. 5,120,548 describes another suitable process for forming coatings from a mixture of a water-insoluble polymer and a leachable water-soluble additive. The porous membranes may also be formed by the addition of pore-formers as disclosed in U.S. Pat. No. 4,612,008. In addition, vapor-permeable coatings may even be formed from extremely hydrophobic materials such as polyethylene or polyvinylidene difluorid that, when dense, are essentially water-impermeable, as long as such coatings are porous. Materials useful in forming the coating include but are not limited to various grades of acrylic, vinyls, ethers, polyamides, polyesters and cellulosic derivatives that are water-permeable and water-insoluble at physiologically relevant pHs, or are susceptible to being rendered water-insoluble by chemical alteration such as by crosslinking. Nonlimiting examples of suitable polymers (or crosslinked versions) useful in forming the coating include plasticized, unplasticized and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxiated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes. In various embodiments, the coating agent comprises a cellulosic polymer, in particular cellulose ethers, cellulose esters and cellulose ester-ethers, i.e., cellulosic derivatives having a mixture of ester and ether substituents, the coating materials are made or derived from poly(acrylic) acids and esters, poly(methacrylic) acids and esters, and copolymers thereof; the coating agent comprises cellulose acetate, the coating comprises a cellulosic polymer and PEG, the coating comprises cellulose acetate and PEG.

Coating is conducted in conventional fashion, typically by dissolving or suspending the coating material in a solvent and then coating by dipping, spray coating or by pan-coating. In certain embodiments, the coating solution contains 5 to 15 wt % polymer. Typical solvents useful with the cellulosic polymers mentioned above include but are not limited to acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described, for example, in U.S. Pat. No. 5,612,059. Coatings may also be hydrophobic microporous layers wherein the pores are substantially filled with a gas and are not wetted by the aqueous medium but are permeable to water vapor, as disclosed, for example, in U.S. Pat. No. 5,798,119. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Hydrophobic microporous coating materials include but are not limited to polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed, for example, in U.S. Pat. No. 4,247,498, U.S. Pat. No. 4,490,431 and U.S. Pat. No. 4,744,906. Osmotic controlled-release devices may be prepared using procedures known in the pharmaceutical arts. Sec for example, Remington: The Science and Practice of Pharmacy, 20th Edition, 2000.

As further noted above, the agents described herein may be provided in the form of microparticulates, generally ranging in size from about 10 μM to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes** known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size. Other excipients, such as a binder (e.g., microcrystalline cellulose), may be blended with the agent to aid in processing and forming the multiparticulates. In the case of wet granulation, a binder such as microcrystalline cellulose may be included in the granulation fluid to aid in forming a suitable multiparticulate. See, for example, Remington: The Science and Practice of Pharmacy, 20″Edition, 2000. In any case, the resulting particles may themselves constitute the therapeutic composition or they may be coated by various film-forming materials such as enteric polymers or water-swellable or water-soluble polymers, or they may be combined with other excipients or vehicles to aid in dosing to patients.

### Kits

The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein. The kit may optionally comprise a second pharmaceutical composition comprising one or more additional agents including but not limited to an antibiotic. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Exemplification

### Effects of Dithiothreitol on Stability of the Escherichia coli Heat Stable Enterotoxin

Escherichia coli heat stable enterotoxin (STa; CCELCCNPACTGCY (SEQ ID NO: 1)) binds with high affinity to the receptor guanylate cyclase C (GC-C) located in the membrane of the epithelial cells. When fully folded, SEQ ID NO:1 includes three disulfide bonds (between Cys₁ and Cys₆, between Cys₂ and Cys₁₀ and between Cys₅ and Cys₁₃). To the examine the effects of DTT on SEQ ID NO:1 stability, peptide (0.068 mM) was incubated in 100 mM Tris-HCl pH 8, with various concentrations of dithiothreitol (DTT) from 0.005 to 5 mM for 30 minutes at 37° C. After incubation, free thiols were alkylated by adding iodoacetamide to 50 mM followed by incubation at room temperature for 1 hour in the dark. The reduced and alkylated peptide was then diluted five-fold in 0.1% formic acid in water and purified from the reaction using solid phase extraction in C18 minicolumns (Amprep C18 100 mg). Aliquots of the reaction mixture were applied to an Atlantis dC18 2.1×50 mm column (Waters), equilibrated in 98% buffer A (0.1% formic acid), 2% buffer B (0.1% formic acid, 85% methanol, 15% acetonitrile) at a flow rate of 0.3 ml/min. After a 4 min wash with the same buffers, peptide was eluted with a linear gradient of 2% to 40% buffer B over 38 min with a constant flow rate of 0.3 mL/min. Peptide mass was detected using a Micromass Q-T of 2 instrument equipped with an electrospray ionization (ESI) source operating in positive ion mode. LC-TOF/MS (liquid chromatograph/time-of-flight mass spectrometry) data were collected over a mass range of m/z 100 to 1000. Molecular weight predictions and LC-TOF/MS data analysis were determined with MassLynx version 4.0 software.

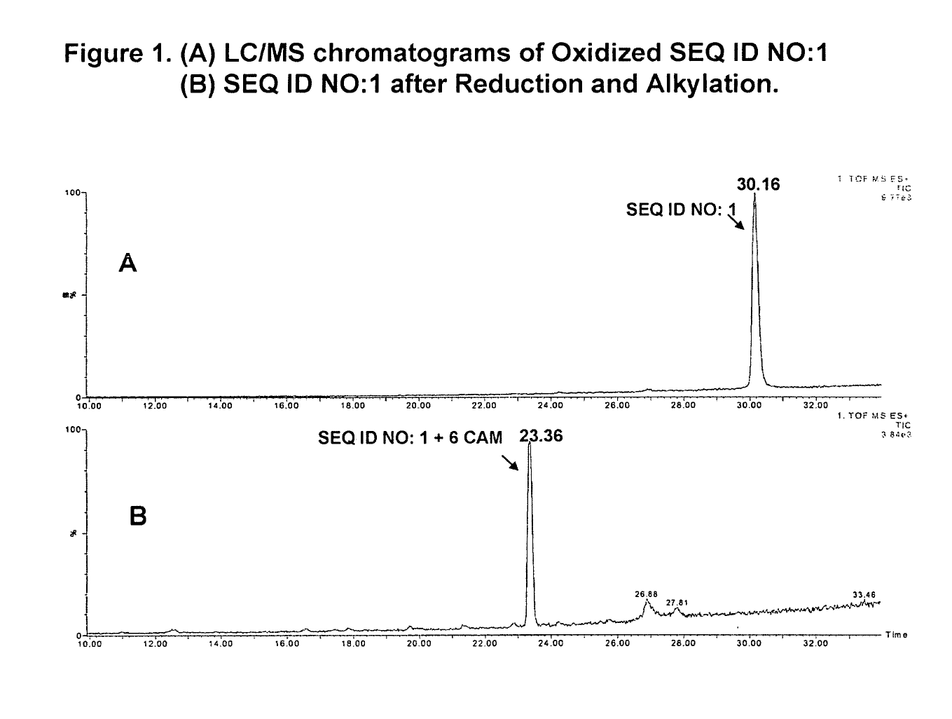

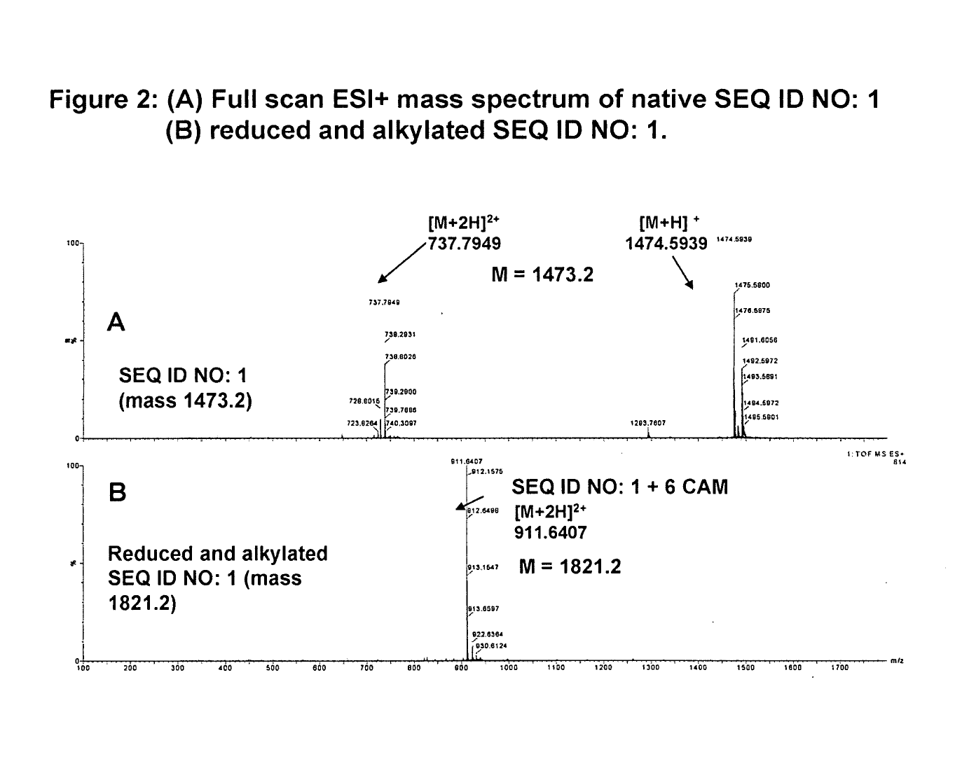

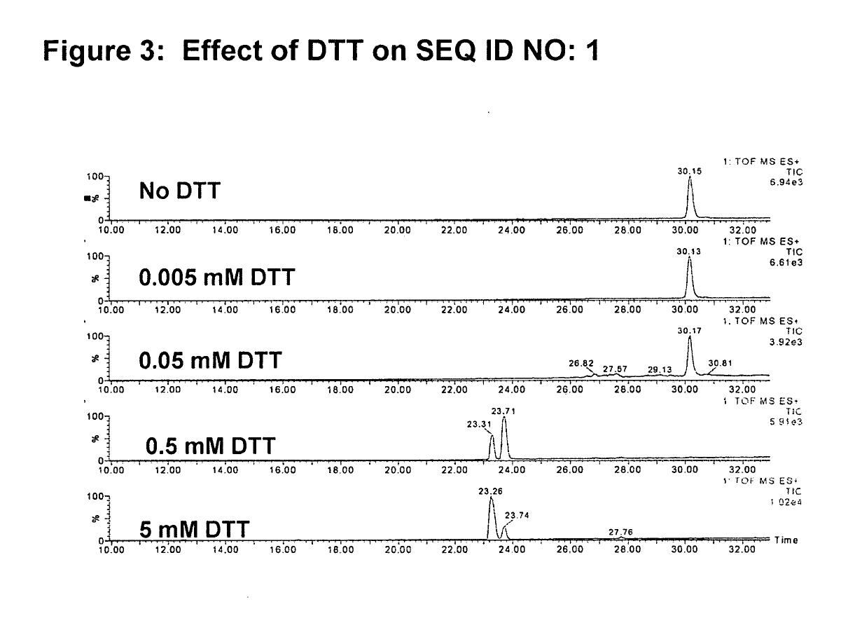

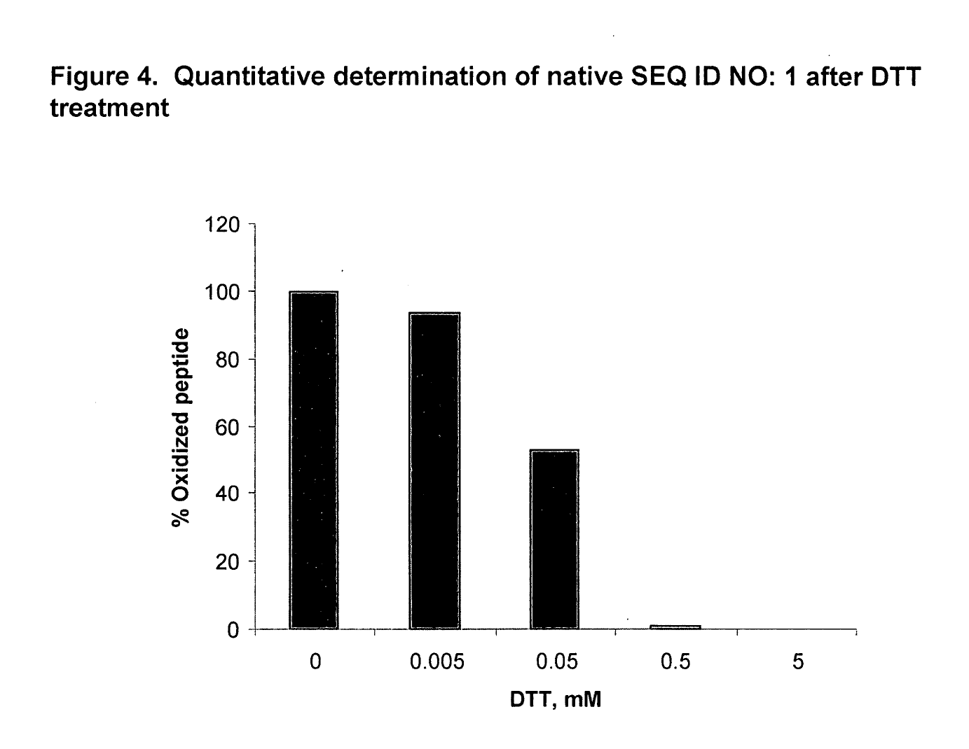

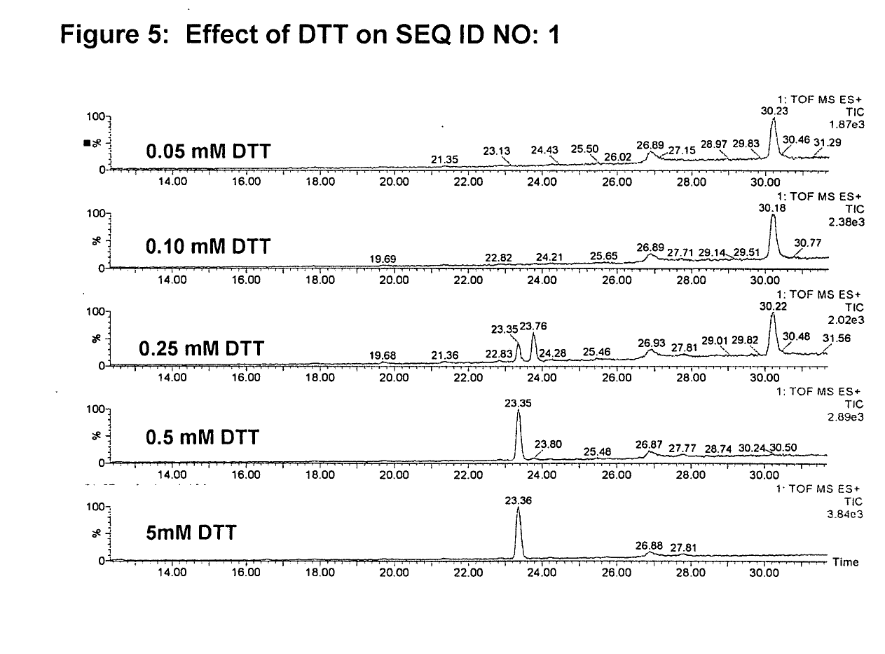

The effect of DTT on SEQ ID NO:1 was analyzed by LC/MS. Reduced cysteines in SEQ ID NO:1 were alkylated by iodoacetamide to form the carboxymethyl amido (CAM) derivatives. Six CAM derivatives were detected making the reduced and alkylated SEQ ID NO:1 elute sooner from a C18 reverse phase HPLC column (23.4 min) than the oxidized native SEQ ID NO:1 (30.2 min) (FIG. 1). Each CAM residue adds a mass of 58 Da to the original mass of oxidized cysteine. The reduced and alkylated SEQ ID NO:1 has the mass of 1821.2 Da (m/z of 911.64 for doubly charged peptide, FIG. 2B), compared to 1473.2 Da for native SEQ ID NO:1 (m/z of 737.8 for doubly charged peptide, FIG. 2A). The difference in mass between native SEQ ID NO:1 and after reduction and alkylation is 384 Da (58 Da for every alkylated sulfhydryl). As shown in FIG. 3, 0.005 mM DTT and 0.05 mM have no visible effect on SEQ ID NO:1. However, based on peak area 0.05 mM DTT results in a reduction of 47% of native SEQ ID NO:1 (FIG. 4). The use of 0.5 mM DTT is equivalent to 1.24 moles of DTT per mole of cysteine residue in SEQ ID NO:1. FIG. 5 shows when SEQ ID NO: 1 is treated with 0.25 mM DTT, a small amount of fully reduced peptide is observed; 0.5 mM and 5 mM DTT cause complete reduction of the disulfide bonds. Thus, SEQ ID NO: 1 is reduced by 0.5 mM DTT (1.3:1 molar ratio DTT to cysteine).

Suckling Mouse Model of Intestinal Secretion (SuMi Assay)

The agents of the present disclosure (e.g. agents that promote the reduction of disulfide bonds) can be tested for their ability to decrease intestinal secretion using a suckling mouse model of intestinal secretion. In this model, a toxin such as the Escherichia coli heat-stable enterotoxin peptide (ST; available from Sigma-Aldrich, St Louis, Mo.) is administered to suckling mice that are between seven and nine days old in order to induce intestinal secretion. Test compounds (e.g. one or more agents that promote the reduction of disulfide bonds) or vehicle only are coadministered (either simultaneously or sequentially) with ST. After the mice are sacrificed, the gastrointestinal tract from the stomach to the cecum is dissected (“guts”). The remains (“carcass”) as well as the guts are weighed and the ratio of guts to carcass weight is calculated. The ability of agents that promote reduction of disulfide bonds to decrease intestinal secretion is reflected in their ability to decrease the ratio of guts to carcass. In certain cases, the ratio for ST alone or ST and vehicle only may be 0.09 or above.

Murine Gastrointestinal Transit (GIT) Assay

In order to determine whether agents of the present disclosure (e.g. agents that promote the reduction of disulfide bonds) decrease the rate of gastrointestinal transit caused by toxin administration, they can be tested in the murine gastrointestinal transit (GIT) assay (Moon et al. Infection and Immunity 25:127, 1979). In this assay, charcoal, which can be readily visualized in the gastrointestinal tract is administered to mice after the administration of a toxin such as the Escherichia coli heat-stable enterotoxin peptide (ST) coadministered (either sequentially or simultaneously) with vehicle only or with one or more agents that promote the reduction of disulfide bonds. The distance traveled by the charcoal is measured and expressed as a percentage of the total length of the colon.

Mice are fasted with free access to water for 12 to 16 hours before the treatment. Toxin (orally administered at 1 μg/kg-1 mg/kg of toxin in buffer (20 mM Tris pH 7.5)) and either vehicle only or one or more agents that promote the reduction of disulfide bonds are administered seven minutes before being given an oral dose of 5% Activated Carbon (Aldrich 242276-250G). After 15 minutes, the mice are sacrificed and their intestines from the stomach to the cecum are dissected. The total length of the intestine as well as the distance traveled from the stomach to the charcoal front is measured for each animal and the results are expressed as the percent of the total length of the intestine traveled by the charcoal front. Results are reported as the average of 10 mice±standard deviation. A comparison of the distance traveled by the charcoal between the mice treated with toxin and vehicle only versus the mice treated with toxin and one or more agents that promoter the reduction of disulfide bonds is performed using a Student's t test and a statistically significant difference is considered for P<0.05.

Effect on Secretion in Ligated Loops Rodent Models

The effect of agents of the present disclosure (e.g. agents that promote the reduction of disulfide bonds) on secretion are studied by co-injecting (either sequentially or simultaneously) a toxin such as the Escherichia coli heat-stable enterotoxin peptide (ST) and either vehicle alone (e.g. 20 mM Tris, pH 7.5 or Krebs Ringer, 10 mM Glucose, HEPES buffer (KRGH)) or one or more agents that promote the reduction of disulfide bonds into an isolated loop in mice. This is done by surgically ligating a loop in the small intestine of the mouse. The methodology for ligated loop formation is similar to that described in London et al. 1997 μm J Physiol p. G93-105. The loop is roughly centered and is a length of 1-3 cm. The loops are injected with ST and vehicle only or ST and one or more agents that promote the reduction of disulfide bonds. Following a recovery time of 90 minutes the loops are excised. Weights are recorded for each loop before and after removal of the fluid contained therein. The length of each loop is also recorded. A weight to length ratio (W/L) for each loop is calculated to determine the effects of agents that promote the reduction of disulfide bonds on toxin induced secretion.

Protocols similar to those described above to perform the assay in female CD rats. In the case of the rat, however four loops of intestine are surgically ligated. The first three loops are distributed equally in the small intestine and the fourth loop is located in colon. Loops are 1 to 3 centimeters.

In Vivo Assay in Human Subjects

To determine whether agents of the present disclosure (e.g. agents that promote the reduction of disulfide bonds) are effective in preventing, inhibiting, or treating a disease or infection associated with toxin (e.g. enterotoxigenic E. coli infection), assays similar to those described for RBC preparations in United States patent publication, US20050158284 (paragraphs 54-65 are herein incorporated by reference) can be undertaken.

Histology Effects

Histology analysis is performed to determine whether agents of the present disclosure (e.g. agents that promote the reduction of disulfide bonds) are able to prevent and/or treat the effects of toxin-associated damage (e.g. atrophy of intestinal epithelial cells) on epithelial cytoarchitecture. Animals (e.g. rodents such as mice or rats, primates, etc.) are administered toxin (e.g. ST toxin) in combination (either sequential or simultaneous administration) with either vehicle only or one or more agents that promote the reduction of disulfide bonds. In the case of prevention, vehicle only or one or more agents that promote the reduction of disulfide bonds are administered before (e.g. single or multiple administration(s) 30 minutes-24 hours or 1-5 days) toxin administration. Animals can be sacrificed at various time points and histological analysis performed.

1-59. (canceled)

60. A method for treating a patient that is: a) intoxicated with a toxin containing at least one disulfide bond or a toxin containing at least one heavy metal and/or b) infected with a microorganism that produces a toxin containing at least one disulfide bond or a toxin containing at least one heavy metal, the method comprising:
administering to the patient a composition comprising an agent or combination of agents that promotes reduction of disulfide bonds or metal ion binding.

61. The method of claim 60, wherein the toxin or the microorganism is present in the digestive tract or airways.

62. The method of claim 60, wherein the toxin comprises a peptide.

63. The method of claim 62, wherein the peptide contains at least one interchain or intrachain disulfide bond.

64. The method of claim 60, wherein the toxin is not a peptide.

65. The method of claim 60, wherein the toxin requires the binding of a metal ion for activity.

66. The method of claim 60, wherein the administration of the composition is selected from enteral administration, oral administration, rectal administration, topical administration, nasal administration, inhalation administration, and dermis application.

67. The method of claim 60, wherein the patient is infected with a microorganism that produces the toxin.

68. The method of claim 67, wherein the microorganism is a bacteria.

69. The method of claim 68, wherein the bacteria is selected from: Escherichia coli, Vibrio cholerae, Clostridium species, Campylobacter species, Shigella species, Pseudomonas species, Bordetella species, and Salmonella species.

70. The method of claim 60, wherein the toxin is a scorpion toxin.

71. The method of claim 70, wherein the scorpion toxin is a specific inhibitor of sodium channels or potassium channels.

72. The method of claim 60, wherein the patient is selected from a child, an infant, immunocompromised individual, elderly individual, individual suffering from diarrhea, individual suffering from food poisoning, and victim of bioterrorism.

73. The method of claim 60 further comprising administering an antibiotic or analgesic.

74. The method of claim 73, wherein the antibiotic is within a class of antibiotics selected from: Anthracyclines; Aminoglycosides; Carbapenems; Carbacephems; Cephamycins; Glycopeptides; Ketolides; Macrolides; Oxacephems; Penicillins; Polymyxins; Quinolones; Rifamycins; and Tetracyclines

75. The method of claim 60, wherein the agent is a reducing agent.

76. The method of claim 75, wherein the reducing agent is a disulfide reducing agent.

77. The method of claim 76, wherein the reducing agent is chosen from: cysteine, mercaptoethanol, 2-mercaptoethanol, 2-mercaptoethylamine, dithioerythritol, dithiothreitol, glutathione, Tiopronin, 2-mercaptopropionic acid, n-acetylcysteine, ascorbic acid, stannous ions/salts, sodium bisulphate, alkali metal and alkaline earth metal borohydrides, triacetoxyborohydrides, cyanoborohydrides and dithionites, and the transition metal salts of transition metals such as zinc, iron, and manganese.

78. The method of claim 60, wherein the patient is administered an agent selected from the group consisting of glutathione, glutathione disulfide, glutathione reductase, glutaredoxin, NADPH and NADP.

79. The method of claim 78, wherein the patient is administered a combination of agents that comprises glutathione and glutaredoxin.

80. The method of claim 78, wherein the patient is administered a combination of agents that comprises glutathione plus glutathione reductase.

81. The method of claim 78, wherein the patient is administered a combination of agents that comprises glutaredoxin and glutathione reductase.

82. The method of claim 78, wherein the patient is administered a combination of agents that comprises glutathione or glutathione disulfide, glutaredoxin and glutathione reductase.

83. The method of claim 78, wherein the patient is administered a combination of agents that comprises glutathione disulfide and an agent selected from: riboflavin, niacinamide, selenium, lipoic acid, and glutathione reductase.

84. A pharmaceutical composition comprising an agent or combination of agents that promotes reduction of disulfide bonds and a pharmaceutically acceptable carrier.

85. The pharmaceutical composition of claim 84 further comprising an antibiotic.

86. The pharmaceutical composition of claim 85 wherein the antibiotic is within a class of antibiotics selected from: Anthracyclines; Aminoglycosides; Carbapenems; Carbacephems; Cephamycins; Glycopeptides; Ketolides; Macrolides; Oxacephems; Penicillins; Polymyxins; Quinolones; Rifamycins; and Tetracyclines

87. The pharmaceutical composition of claim 84 further comprising an analgesic.

88. The pharmaceutical composition of claim 84, wherein the agent is selected from:
cysteine, mercaptoethanol, 2-mercaptoethanol, 2-mercaptoethylamine, dithioerythritol, dithiothreitol, glutathione, Tiopronin, 2-mercaptopropionic acid, n-acetylcysteine, ascorbic acid, stannous ions/salts, sodium bisulphate, alkali metal and alkaline earth metal borohydrides, triacetoxyborohydrides, cyanoborohydrides and dithionites, and the transition metal salts of transition metals such as zinc, iron, and manganese.

89. The pharmaceutical composition of claim 84 comprising an agent or combination of agents selected from the group consisting of glutathione, glutathione disulfide, glutathione reductase, glutaredoxin, NADPH and NADP.

90. The pharmaceutical composition of claim 89, wherein the combination of agents comprises glutathione and glutaredoxin.

91. The pharmaceutical composition of claim 89, wherein the combination of agents comprises glutathione plus glutathione reductase.

92. The pharmaceutical composition of claim 89, wherein the combination of agents comprises glutaredoxin and glutathione reductase.

93. The pharmaceutical composition of claim 89, wherein the combination of agents comprises glutathione or glutathione disulfide, glutaredoxin and glutathione reductase.

94. The pharmaceutical composition of claim 89, wherein the agent comprises glutathione disulfide and the composition further comprises an agent selected from riboflavin, niacinamide, selenium, lipoic acid, and glutathione reductase.
